(19) United States
(12) Patent Application Publication
(10) Pub. No.: US 2022/0218517 A1
(43) Pub. Date: Jul. 14, 2022

(54) Health Care Device

(71) Applicant: Cheng-Chuan Yang, Taichung City (TW)

(72) Inventor: Cheng-Chuan Yang, Taichung City (TW)

(21) Appl. No.: 17/145,397

(22) Filed: Jan. 11, 2021

(51) Int. Cl. A61F 7/02; A61F 7/00

(52) U.S. Cl. CPC A61F 7/0241; A61F 7/0085; A61F 2007/0054; A61F 2007/0012; A61F 2007/0004

(57) ABSTRACT

A health care device includes a soft water bag, a control unit disposed on a side of the soft water bag, a temperature control element electrically connected to the control unit and disposed inside the soft water bag. When the control unit controls the temperature control element, the temperature control element disposed inside the soft water bag is energized to heat and control a temperature of liquid in the soft water bag, so as to provide better portability than the conventional products.

### BACKGROUND OF THE INVENTION

### 1. Field of the Invention

The present invention is related to a health care device, more particularly to a health care device capable of applying hot compression on a body part, which needs to be massaged, so as to achieve the effect of relieving fatigue.

### 2. Description of the Related Art

In order to relieve the fatigue and discomfort of eyes, besides properly resting the eyes, applying heat compression on the muscles around body part can also relieve soreness and discomfort of eyes.

Please refer to Taiwan Patent No. M530153, which discloses an eye massager, as shown in FIGS. 1 to 8 of the aforementioned patent, the eye massager includes a main body, a water bag, a control unit, a heating element, a circulating water tank, a circulating tube, a heating tube and a cover housing. The circulation tube is inserted through the main body and connected between a circulating pump and a diversion channel of the water bag, and connected between the circulating water tank and the diversion channel of the water bag. The control unit is electrically connected to the circulating pump of the main body and the heating element, after the control unit receives the sensed temperature of the circulating water tank, the control unit can drive the heating element to increase or stop increasing the temperature to achieve the purpose of temperature control.

However, the aforementioned eye health care device still has shortcomings.

The eye massager can use the control unit to drive the heating element to increase or stop increasing the temperature, and use the circulating pump to circulate the liquid flowing between the circulating water tank and the water bag to achieve the effect of temperature control. However, the body, the control unit, the heating element, the circulating water tank are accommodated inside cover housing, it results in the large volume of the eye health care device, and it is difficult for a user to carry the eye health care device of large volume.

Therefore, in order to solve the conventional technical problem, the inventors spend a lot of time in researching relevant knowledge, and comparing the pros and cons, conducting research and development of related products, and many experiments and tests, to develop a health care device.

### SUMMARY OF THE INVENTION

The present invention provides a health care device including a soft water bag, a control unit disposed on a side of the soft water bag, and a temperature control element electrically connected to the control unit and disposed inside the soft water bag. When the control unit controls the temperature control element, the temperature control element disposed inside the soft water bag is energized to heat and control a temperature of liquid in the soft water bag.

The above-mentioned water bag can be attached on the body part, the microprocessor of the control unit can control and drive the temperature control element to increase the temperature, so as to promote blood circulation of the body part, relieve soreness and discomfort, and solve the conventional problem that size of the conventional eye massager is too large to carry.

### BRIEF DESCRIPTION OF THE DRAWINGS

The structure, operating principle and effects of the present invention will be described in detail by way of various embodiments which are illustrated in the accompanying drawings.

FIG. 1 is a perspective view of a health care device of a first embodiment, according to the present invention.

FIG. 2 is a schematic view showing that a health care device of the first embodiment is attached on a user's leg.

FIG. 3 is a perspective view of a health care device of a second embodiment, according to the present invention.

FIG. 4 is a schematic view of FIG. 3 when viewed from another angle.

FIG. 5 is a schematic view showing that the health care device of the second embodiment is attached on a user's eyes.

FIG. 6 is a perspective view of a health care device of a third embodiment, according to the present invention.

FIG. 7 is a schematic view showing that the health care device of the third embodiment is attached on a user's neck, according to the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include”, and variations such as “comprises”, “comprising”, “includes”, or “including”, will be acknowledged to imply the inclusion of stated elements but not the exclusion of any other elements.

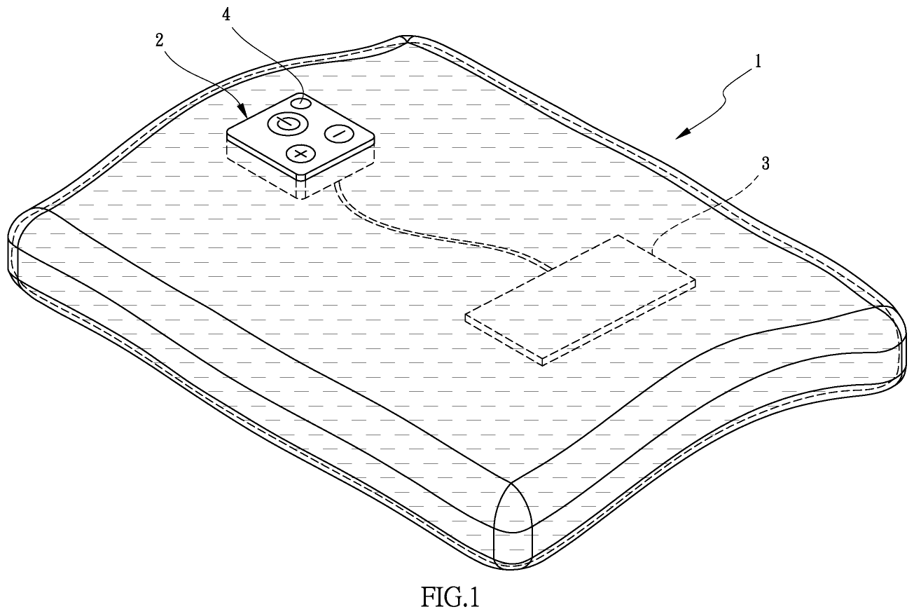

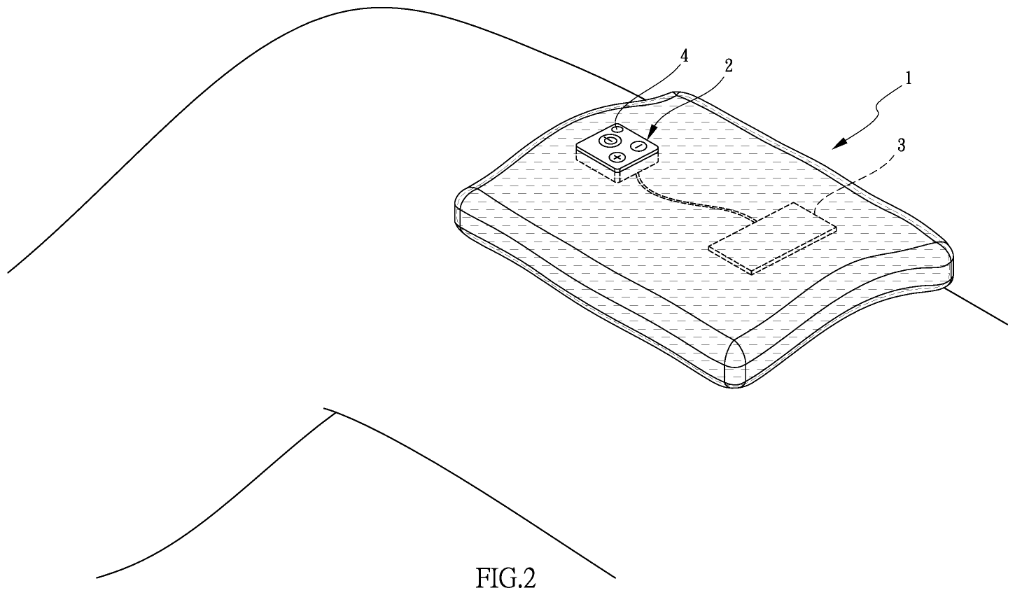

Please refer to FIGS. 1 and 2, which show a health care device of a first embodiment of the present invention. The health care device includes a soft water bag 1, a control unit 2 disposed on a side of the soft water bag 1, and a temperature control element 3 electrically connected to the control unit 2 and disposed inside the soft water bag 1. When the control unit 2 controls the temperature control element 3, the temperature control element 3 disposed inside the soft water bag 1 is energized to heat and control a temperature of liquid in the soft water bag 1.

According to aforementioned content, the health care device uses the control unit 2 to control and drive the temperature control element 3 located in the water bag to energize for heating, when the heated soft water bag 1 is attached on a user's body part, the soft water bag 1 can perform hot compression on the attached body part, as shown in FIG. 2, which shows the soft water bag 1 is attached on the user's leg part as exemplary illustration. As a result, the heated soft water bag 1 can promote blood circulation and relieve fatigue and the soreness of the part.

The temperature control element 3 of the present invention is formed by thermal conduction film including high thermal conductive material such as copper film, aluminum film or graphite plate, and the temperature control element 3 is electrically connected to the control unit 2, and insulation material such as a PET film or a silicon rubber is covered on the surface of the temperature control element 3 to form a layer of insulation film which is not shown in figures, so as to prevent the temperature control element 3 from contacting the liquid in the soft water bag 1 to cause short circuiting during the process of energizing to heat. The heat generated by energization can be conducted to the liquid in the soft water bag 1 through the temperature control element 3, to increase the temperature of the soft water bag 1.

The health care device can include a temperature sensor 4 disposed on a surface of the control unit 2 and electrically connected to the control unit 2. The health care device can be energized to heat up to 45° C., and the temperature of 45° C. is set as a threshold for the high-temperature hot compression. When the temperature sensor 4 senses that the temperature reaches 45° C., the control unit 2 obtains the sensing information indicative of 45° C. and stops driving the temperature control element 3. When the temperature sensor 4 senses that the temperature is lower than 35° C., the control unit 2 obtains the sensing information indicative of lower than 35° C. and drives the temperature control element 3 to increase temperature, so that the temperature of the soft water bag 1 can be increased up to 45° C., to remain stable hot compression effect. Besides the aforementioned manner of using the temperature sensor 4 to automatically adjust temperature, the user can use a button disposed on the surface of the control unit 2 to control temperature manually, to adjust the temperature of the soft water bag 1 to an appropriate temperature.

Furthermore, when the control unit 2 fails to obtain the sensing information, the control unit 2 is determined as abnormal, and the temperature control element 3 is forced to stop increasing the temperature, to prevent from damage of the temperature sensor 4 or prevent from occurring safety accident due to failure of transmitting the signal indicative of sensed temperature.

The soft water bag 1 of the health care device is made by plastic material such as silica gel, rubber or PVC, and the plastic property of the soft water bag 1 provides the soft water bag 1 to snugly attach with the body part.

The details of a second embodiment and a third embodiment are described in the following paragraphs.

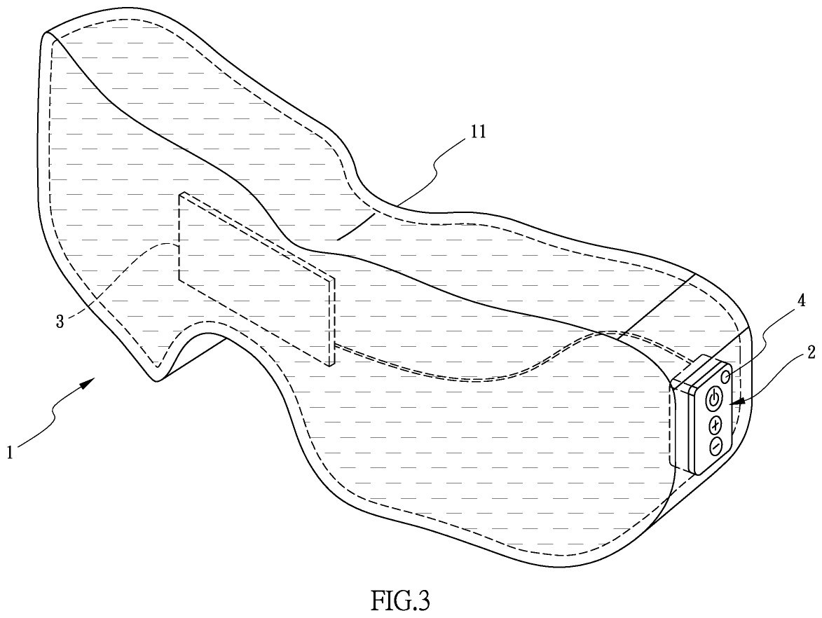

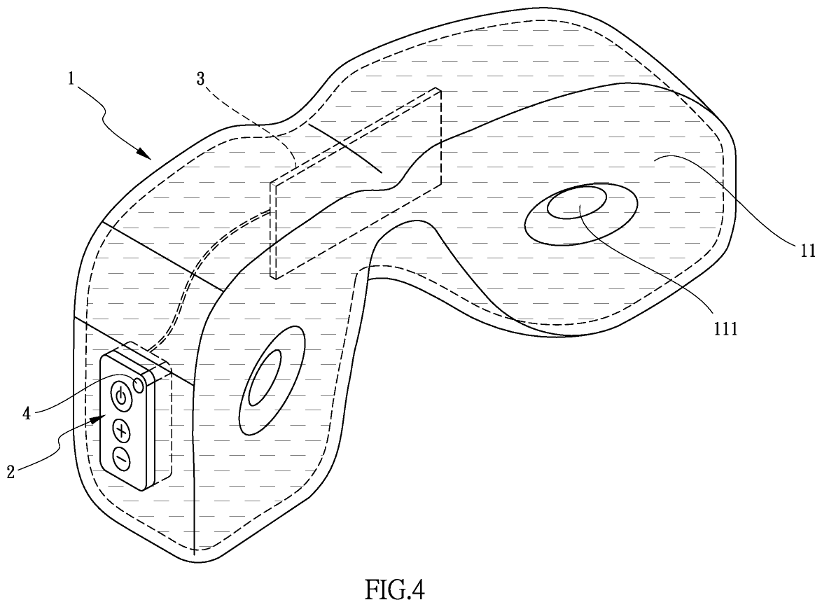

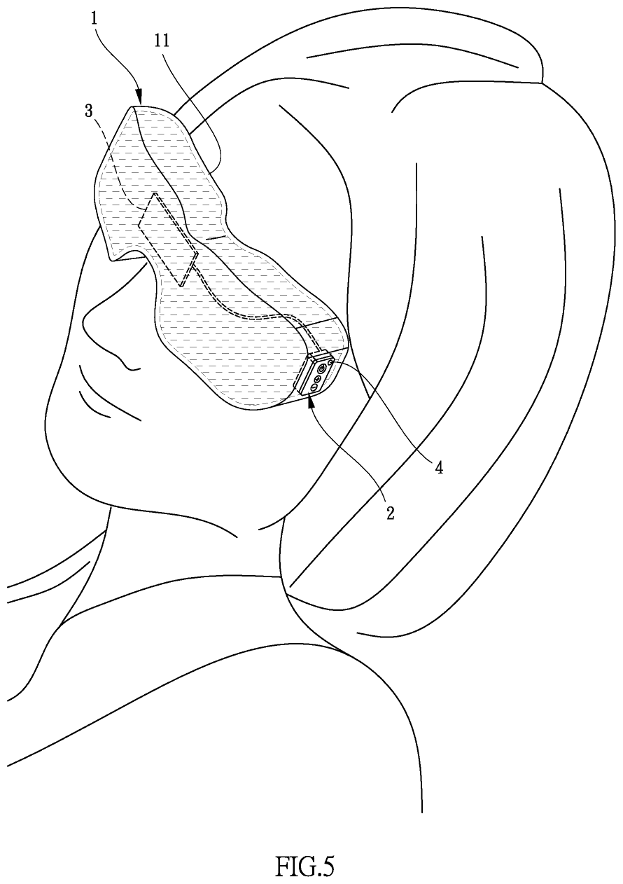

As shown in FIGS. 3 to 5, the difference between the second embodiment and the first embodiment is that the soft water bag 1 of the second embodiment is in an elongate shape and includes a pasted part 11 formed on a convex side thereof, the pasted part 11 includes two eye avoidance grooves 111 recessed thereon, and when the pasted part 11 of the soft water bag 1 are attached on user's eyes, the eyes can be accommodated in the eye avoidance groove 111, to prevent the eyes from in direct contact with the soft water bag 1 or being too close to the soft water bag 1, thereby reducing the defective effect of hot compression on the eyes, for example, the eyes are burnt by excessively high temperature, or too high temperature causes transient blindness. Furthermore, the pasted part 11 has a certain thickness to appropriately block heat, so that the user's eye and skin surrounding the eyes can be prevented from being indirectly burned by the high-temperature liquid in the soft water bag 1.

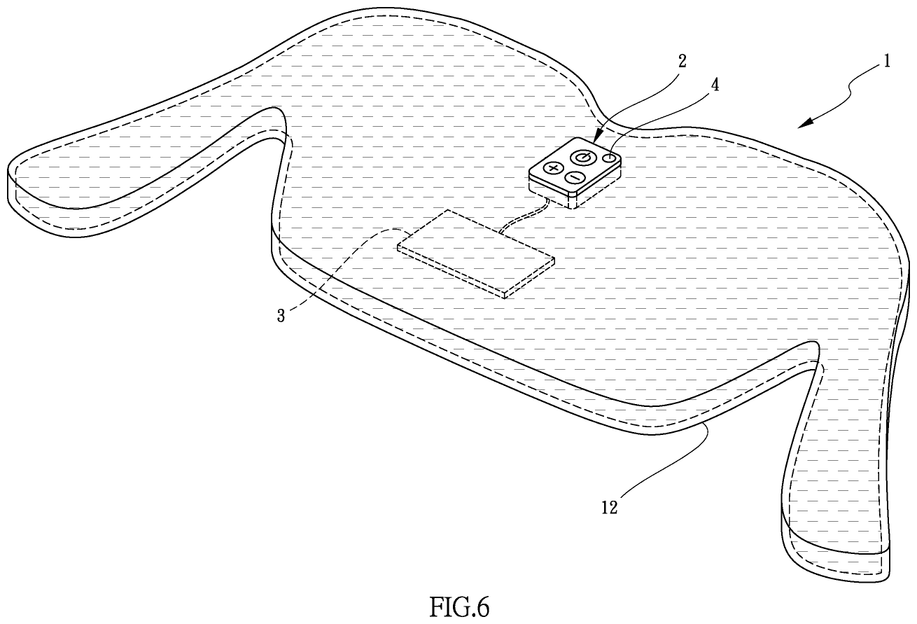

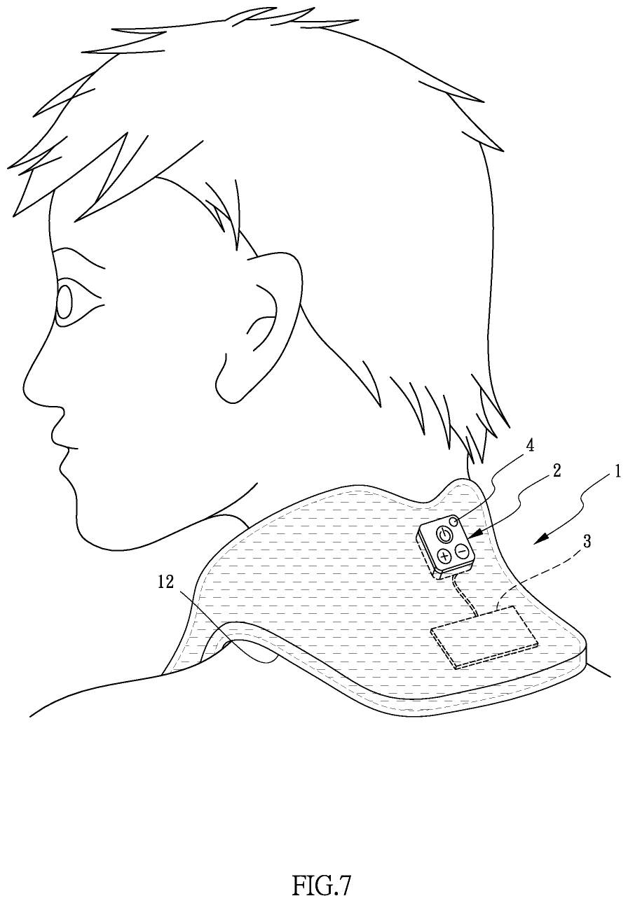

As shown in FIGS. 6 and 7, the difference between the third embodiment and the first embodiment is that when the soft water bag 1 includes a neckrest part 12 disposed on other side of the soft water bag opposite to the control unit 2, so that when the soft water bag 1 is attached with the user, the soft water bag 1 can attached with the user's neck part.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A health care device, comprising:
a soft water bag;
a control unit, disposed on a side of the soft water bag; and
a temperature control element, electrically connected to the control unit and disposed inside the soft water bag, wherein when the control unit controls the temperature control element, the temperature control element disposed inside the soft water bag is energized to heat and control a temperature of liquid in the soft water bag.

2. The health care device according to claim 1, wherein the soft water bag is made by plastic material comprising silica gel, rubber or polyvinyl chloride.

3. The health care device according to claim 1, further comprising a temperature sensor disposed on a surface of the control unit, and electrically connected to the control unit.

4. The health care device according to claim 1, wherein the soft water bag is in an elongate shape and comprises a pasted part configured on a convex side thereof, and the pasted part comprises two eye avoidance grooves recessed thereon.

5. The health care device according to claim 1, further comprising a neckrest part configured to attach, wherein the neckrest part is disposed on other side of the soft water bag opposite to the control unit.

6. The health care device according to claim 1, wherein the temperature control element is made by high thermal conductivity material including copper film, aluminum film or graphite plate.
